(19) United States
(12) Patent Application Publication
(10) Pub. No.: US 2013/0116716 A1
(43) Pub. Date: May 9, 2013

(54) LIQUID-JET SCALPEL

(71) Applicant: Deutsches Zentrum fuer Luft-und Raumfahrt e.V., Cologne (DE)

(72) Inventors: Thomas Bahls, Weil (DE); Florian Alexander Froehlich, Germering (DE); Sophie Lantermann, Muenchen (DE)

(73) Assignee: DEUTSCHES ZENTRUM FUER LUFT-UND RAUMFAHRT E.V., Cologne (DE)

(21) Appl. No.: 13/670,998

(22) Filed: Nov. 7, 2012

(30) Foreign Application Priority Data
Nov. 9, 2011 (DE) 102011086032.0

(51) Int. Cl. A61B 17/3203

(52) U.S. Cl. CPC A61B 17/3203
USPC 606/167

(57) ABSTRACT

A liquid-jet scalpel for minimally invasive surgery, comprising a nozzle for ejecting a liquid jet, and a functional end effector for manipulating tissue at the surgical site.

### CROSS REFERENCE TO RELATED APPLICATIONS

The present invention claims the priority of German Patent Application no. DE 10 2011 086 032.0 filed on Nov. 9, 2011, the disclosures of which are incorporated herein by reference.

### BACKGROUND OF THE INVENTION

1. Field of the Invention

The invention relates to a liquid-jet scalpel for minimally invasive surgery.

2. Description of the Prior Art

In contrast to open surgery, minimally invasive surgery manages without large cuts such as, for instance, opening the thorax of a patient. Instead, only small cuts are made via which cameras and instruments such as scalpels, scissors, forceps and the like, are introduced into the body with the help of trocars. With these instruments and with the use of the camera, the respective surgical intervention is performed. However, surgery via the trocar points entails the loss of two degrees of freedom.

Further, it is known from prior art to use liquid-jet scalpels, e.g. water-jet scalpels, in open surgery. Among others, it is one advantage of water-jet scalpels that they can be used to selectively cut tissue. This means that the pressure of the water jet used can be adjusted such that only the targeted tissue is cut or ablated. For example, it is possible to ablate only affected cells by means of the water jet, while healthy cells remain substantially intact. It is also possible to cut or ablate soft tissue (such as the liver), for example, and to simultaneously spare the blood vessels and nerves so that no loss of blood or only a little loss of blood occurs during the intervention. Thus, the water-jet scalpel can reduce a patient's surgical trauma.

In minimally invasive surgery it is further desirable to keep the number of necessary changes of instruments during surgery as low as possible.

### SUMMARY OF THE INVENTION

It is an object of the invention to provide a liquid-jet scalpel for minimally invasive surgery with which the number of necessary changes of instruments during surgery can be reduced.

The present liquid-jet scalpel for minimally invasive surgery comprises a nozzle for ejecting a liquid jet. The preferred liquid used is water, while, however, other appropriate liquids could be used as well.

According to the invention, the liquid-jet scalpel is provided, in addition to the nozzle, with a functional end effector for manipulating tissue at the surgical site. This functional end effector thus is a component separate from the nozzle for ejecting the liquid jet.

This functional end effector may be, for example, a gripper, a pair of scissors or a stapler, each with or without an integrated mono- or bipolar electrocauter. Other tools used in minimally invasive surgery are also conceivable.

Therefore, the liquid-jet scalpel of the present invention allows cutting tissue with the liquid jet emanating from the nozzle and, in an immediately successive, previous or even simultaneous step, allows manipulating tissue at the surgical site with the functional end effector without the liquid-jet scalpel having to be extracted from the patient's body to replace it with another instrument. The liquid-jet scalpel may thus remain in the trocar used. Thereby, it is possible, according to the invention, on the one hand, to treat tissue at the surgical site using the liquid jet and, on the other hand, to manipulate tissue using the functional end effector. An intricate and time-consuming change of instruments during surgery can thus be avoided.

It is preferred that the liquid-jet scalpel has a suction opening at its distal end for sucking the liquid and the detached tissue from the surgical site. The suction device is thus provided in addition to the nozzle and the functional end effector at the distal end of the liquid-jet scalpel.

It is further preferred that the nozzle is fastened at the distal end of the liquid-jet scalpel through a joint providing in particular at least one degree of freedom and preferably at least two degrees of freedom. Thereby, the degrees of freedom can be compensated that are lost due to the use of the trocar points in minimally invasive surgery. Further details of this feature are described in this application in the context of the second independent invention. The first invention may include all features described in the context of the at least one joint of the second invention.

It is further preferred that the end effector comprises two end effector arms. If the end effector is configured as a pair of scissors, for example, each of the blades of the pair of scissors is respectively referred to as an end effector arm according to the invention. The nozzle may be arranged centrally between the two end effector arms. In this case, the nozzle can extend through the joint by which the shaft is connected with the distal end of the liquid-jet scalpel. Thus, the direction of the liquid jet can be changed by pivoting the joint. Preferably, at least one liquid line extends through the joint, at whose distal end the nozzle is mounted so that the nozzle can be pivoted with the joint and the direction of the liquid jet can be changed thereby.

In a preferred embodiment the end effector has two end effector arms. Here, the suction device is arranged immediately at one end effector arm and the nozzle is arranged at the other end effector arm. The nozzle and the suction device can therefore be used with some space between them.

As an alternative, it is possible to arrange the nozzle and the suction device at the same end effector arm.

In another embodiment the end effector may comprise two end effector arms that each have a spreader element. Thereby, tissue at the surgical site can be pushed apart by spreading the end effector arms. Normally, this would have to be performed with another instrument which, according to this embodiment of the invention, can thus be omitted. For example, the spreader element may be formed such that each end effector arm has a lateral enlargement or thickening by which tissue can be pushed apart.

In another preferred embodiment the nozzle and the suction device, when not used, can be retracted in the proximal direction of the liquid-jet so that they do not hinder the movement of the end effector. Thereby, it is possible to achieve a better manipulation of tissue with the end effector.

In another preferred embodiment the nozzle and/or the suction device extend past the joint, and in particular past a shaft the distal end of the end effector, at the distal end of which the end effector is attached. Thus, they do not extend through the joint or the shaft. In this embodiment, the nozzle and/or the suction device may have a roughened surface at their distal end so that they can be gripped and oriented reliably by the end effector.

It is further preferred that the nozzle and the suction device are arranged coaxially relative to each other and are formed rotationally symmetrical. The joint connecting the shaft with the distal end of the liquid-jet scalpel thus only needs one degree of freedom. At the distal end, the rotation about the shaft axis provides for the two necessary degrees of freedom at the instrument.

A second, independent invention refers to a liquid-jet scalpel that is generally similar to the above described liquid-jet scalpel. The essential feature of the second invention is that the nozzle is connected with the liquid-jet end effector via at least one joint. The term “liquid-jet end effector” refers to the distal end of the liquid-jet scalpel that terminates in the nozzle for ejecting the liquid jet.

In a minimally invasive surgical intervention, during which the liquid-jet end effector is within the body of a patient, the nozzle is thus pivotable within the body of the patient about the at least one joint.

Thus, according to the invention, the at least one additional joint provides at least one additional degree of freedom so that the space for manipulation, in which the liquid-jet scalpel can operate within the body of a patient, is enlarged. It can thus be avoided to withdraw the liquid-jet scalpel, e.g. in order to attach another surgical instrument thereto so as to reach another site in the patient's body. Rather, the liquid-jet scalpel of the present invention allows reaching various parts of the surgical site with a single instrument. Moreover, additional working trocars become obsolete due to the larger working space. This larger working space is due to the additional degrees of freedom of the liquid-jet end effector.

According to the invention, the liquid-jet end effector may be formed exclusively by the nozzle and the at least one joint. As an alternative, it is possible that the liquid-jet end effector has another element proximal of the at least one joint, which element itself is connected with a shaft, for example, via which the liquid-jet end effector is inserted into the body of a patient via a working trocar. Basically, a liquid-jet end effector is characterized in that is can be moved by differently designed kinematics and, depending on the application, comprises a different liquid outlet opening or a nozzle.

Besides its application in minimally invasive surgery, the liquid-jet scalpel can also be used in open surgery, where it also offers the advantage that the at least one additional joint provides additional degrees of freedom in the region of the nozzle.

It is preferred that the liquid-jet scalpel comprises a fastening device for fastening to a medical robot so that the liquid-jet scalpel can be operated via the medical robot. As an alternative, the liquid-jet scalpel may be formed integrally with the medical robot. The use of a medical robot allows applying higher water pressure on the liquid-jet scalpel. Further advantages provided by the use of a liquid-jet scalpel in connection with a medical robot are the higher precision attainable, the possibility of performing repetitive movements, as well as the possibility of planning movement paths in advance and of establishing a surface grid.

It is preferred that the at least one joint is a universal joint or a ball joint so that the movement space of the nozzle in the patient's body is extended by two degrees of freedom.

Further, it is possible that the nozzle is connected with the liquid-jet end effector via a plurality of successively arranged joints, in particular ball joints. For example, a so-called “spine kinematics” can be used so that a better access to regions of a patient's body is possible that are difficult to access or have been impossible to access before. Thereby, new fields of application open up for water-jet surgery.

### BRIEF DESCRIPTION OF THE DRAWINGS

A full and enabling disclosure of the present invention including the best mode thereof, enabling one of ordinary skill in the art to carry out the invention, is set forth in greater detail in the following description, including reference to the accompanying drawing in which

FIGS. 1-6 illustrate various embodiments of a liquid-jet scalpel according to the first embodiment,

FIG. 7a illustrates a liquid-jet end effector of a water-jet scalpel according to prior art,

FIGS. 7b, 7c, 7d illustrate the liquid-jet end effector of various embodiments of a liquid-jet scalpel according to the second invention,

FIG. 8 illustrates the liquid-jet end effector of another embodiment of the liquid-jet scalpel according to the second invention.

### DESCRIPTION OF PREFERRED EMBODIMENTS

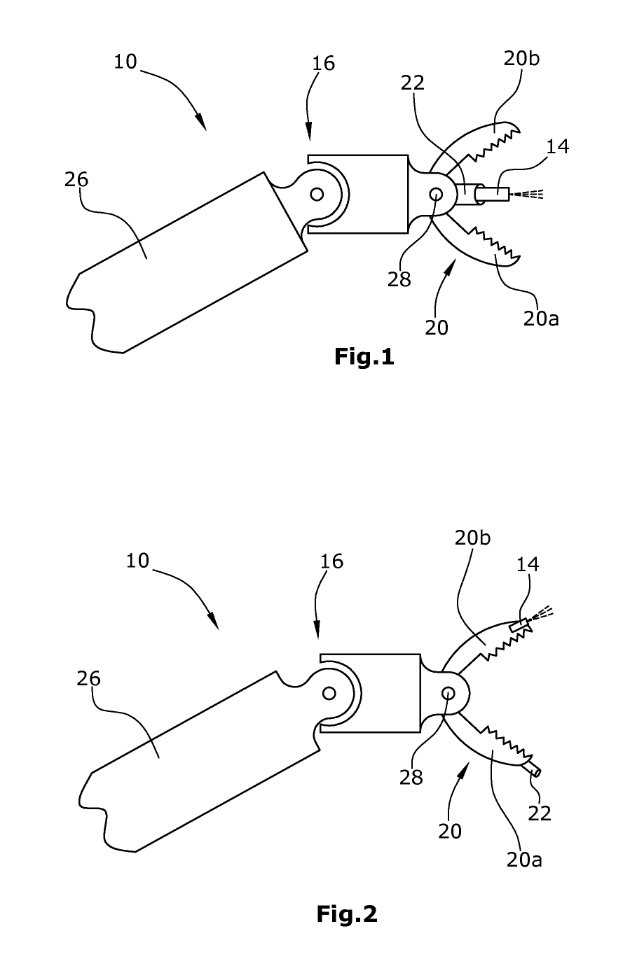

FIGS. 1-6 illustrate features that have already been described in the general part of the description.

Referring to FIG. 1, an embodiment of the present liquid-jet scalpel 10 comprises a shaft 26 which is connected with the functional end effector 20, the nozzle 14 and the suction device 22 via a joint 16. In the present instance, the functional end effector 20 is designed as a gripper and comprises two gripper arms 20a, 20b pivotable relative to each other about a joint 28. As illustrated in FIG. 1, the nozzle 14 and the suction device 22 are arranged between the two gripper arms 20a, 20b. The direction of the liquid jet can be changed by pivoting the nozzle about the joint 16.

Referring to FIG. 2, the suction device 22 is attached to the first gripper arm 20a and the nozzle 14 is attached to the second gripper arm 20b. This allows a more flexible handling of these two instruments in a manner independent of each other.

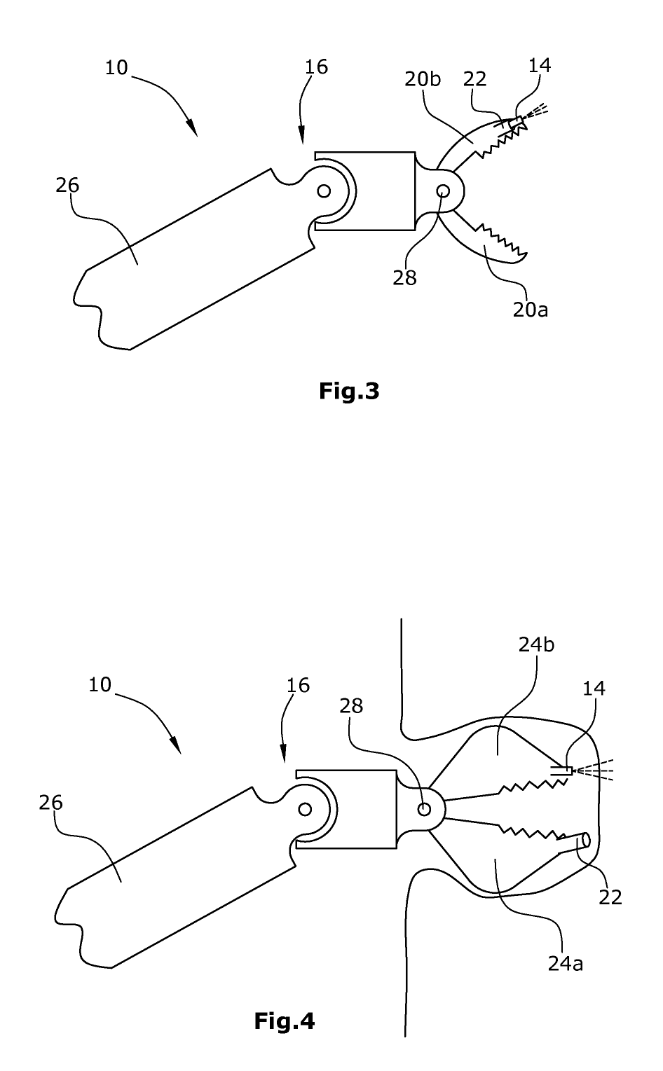

Referring to FIG. 3, the nozzle 14 and the suction device 22 are both arranged at the second gripper arm 20b.

FIG. 4 illustrates an embodiment in which the end effector arms 20a, 20b each comprise a spreader element 24a, 24b. Using the same, tissue at the surgical site can be pushed apart by spreading the gripper arms 20a, 20b.

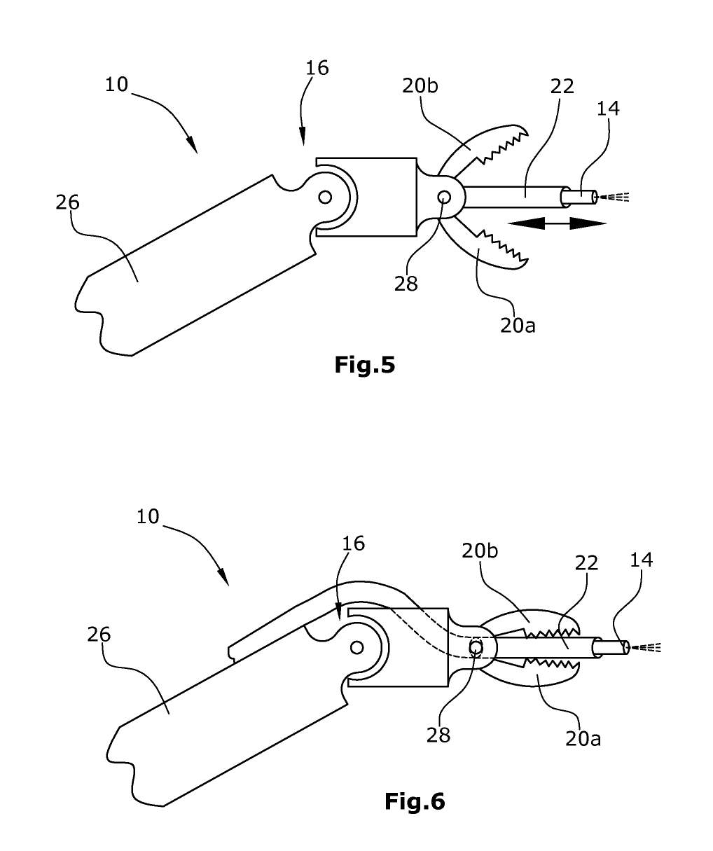

FIG. 5 illustrates an embodiment in which the nozzle and the suction device can be retracted in the proximal direction of the liquid-jet scalpel 10, when they are not used. Thus, they do not impede the movement of the end effector 20.

Referring to FIG. 6, the nozzle 14 and the suction device 22 extend past the joint 16 and the shaft 26. The nozzle 14 and the suction device 22 have a roughened surface at their distal end so that they can be gripped and oriented reliably by the two gripper arms 20a, 20b.

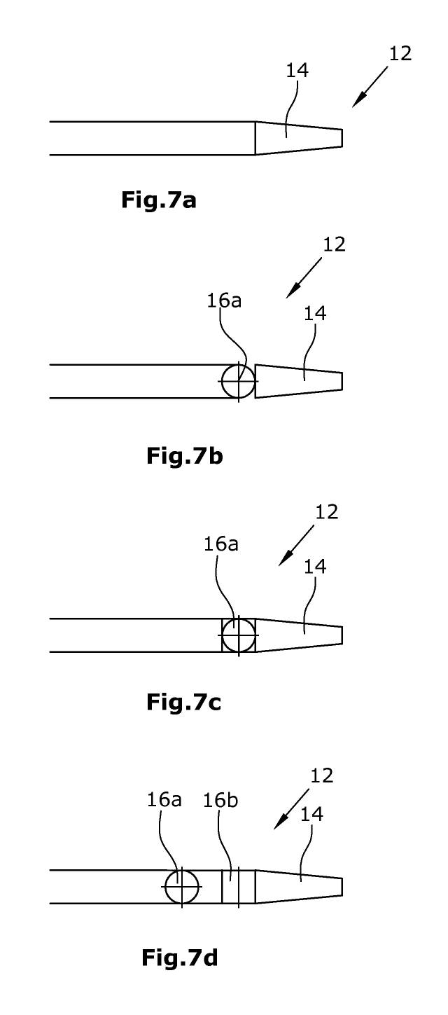

FIG. 7a illustrates an end effector arm of a water-jet scalpel according to prior art. The same is rigidly connected with the nozzle 14.

In contrast, FIGS. 7b, 7c and 7d illustrate different embodiments of a liquid-jet scalpel according to the second invention. In this case, the nozzle 14 is connected with the liquid-jet end effector 12 via a joint 16a so that the nozzle can be pivoted upward and downward with respect to the plane of the drawing sheet. Thus, the nozzle 14 has an additional degree of freedom.

Referring to FIG. 7c, the joint 16a is designed as a joint with two degrees of freedom so that two additional degrees of freedom can be made available. Assuming that the liquid-jet scalpel of FIG. 7a has four degrees of freedom in the surgical space, the liquid-jet scalpel of FIG. 7c offers a total of six degrees of freedom in the surgical space.

The liquid-jet scalpel illustrated in FIG. 7d also allows for six degrees of freedom in the surgical space, wherein at least two successively arranged joints 16a, 16b are used. In this case, the first hinge 16a allows for an upward and downward pivoting of the nozzle, seen in the direction of the drawing sheet, whereas the second joint 16b allows for a pivoting of the nozzle 14 in a direction perpendicular to the sheet.

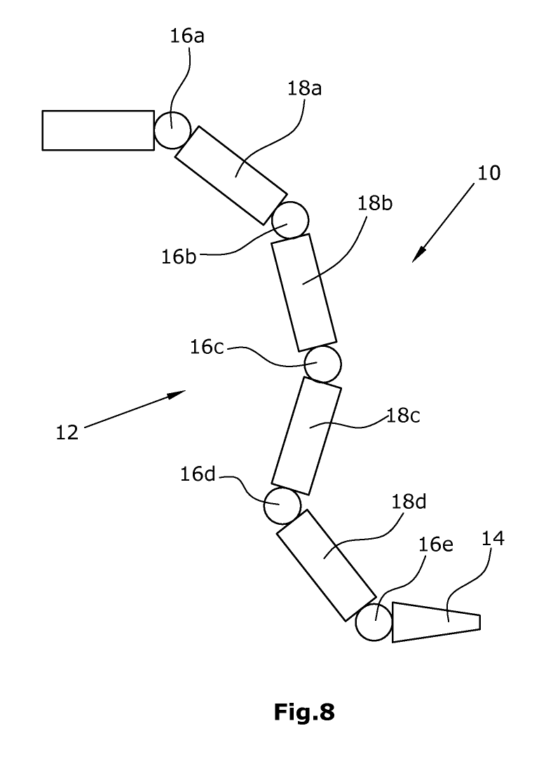

A liquid-jet scalpel 10 with a so-called “spine kinematic” is illustrated in FIG. 8. Here, the liquid-jet end effector 12 is connected with the nozzle 14 via ball joints 16a, 16b, 16c, 16d, 16e. Preferably, connecting elements 18a, 18b, 18c, 18d may be arranged between the ball joints 16a, 16b, 16c, 16d, 16e. Using the liquid-jet scalpel illustrated in FIG. 8, regions in a patient's body that are difficult to access can be reached, it now being possible to reach a determined site in the body in different ways so that, for example, the liquid-jest scalpel 10 can be guided past organs that are in the direct way to the intended surgical site.

The illustrated liquid-jet scalpels of the present invention make it possible to freely determine the position and the orientation of the nozzle 14 so that both the starting point of the incision and the direction of cutting can be determined.

Although the invention has been described and illustrated with reference to specific illustrative embodiments thereof, it is not intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

What is claimed is:

1. A liquid-jet scalpel for minimally invasive surgery, comprising
a nozzle for ejecting a liquid jet, and
a functional end effector for manipulating tissue at the surgical site.

2. The liquid-jet scalpel of claim 1, wherein the functional end effector is a gripper, a pair of scissors, a stapler, each with or without an integrated mono- or bipolar electrocauter or another surgical instrument.

3. The liquid-jet scalpel of claim 1, wherein the liquid-jet scalpel has a suction device at its distal end for sucking liquid and ablated tissue from the surgical site.

4. The liquid-jet scalpel of claim 1, wherein the nozzle is attached to the distal end of the liquid-jet scalpel via a joint having at least one degree of freedom, in particular at least two degrees of freedom.

5. The liquid-jet scalpel of claim 1, wherein the end effector comprises two end effector arms and the nozzle is arranged between these end effector arms, the nozzle in particular extending through the joint and being guided by an element pivotable by means of the joint, so that the direction of the liquid jet can be changed by pivoting the joint.

6. The liquid-jet scalpel of claim 1, wherein the end effector comprises two end effectors and the suction device is arranged at one end effector arm, while the nozzle is provided at the other end effector arm, or the nozzle and the suction device are arranged at the same end effector arm.

7. The liquid-jet scalpel of claim 1, wherein the end effector comprises two end effector arms, each having a spreader element so that tissue at the surgical site can be pushed apart by spreading the end effector arms.

8. The liquid jet scalpel of claim 1, wherein the nozzle and the suction device are adapted to be withdrawn, when not in use, in the proximal direction of the liquid-jet scalpel so that they do not impede the movement of the end effector.

9. The liquid-jet scalpel of claim 1, wherein the nozzle and/or the suction device are guided past the hinge and in particular past a shaft at the distal end of which the end effector is fastened, and thus do not extend through the hinge or the shaft, the nozzle and/or the suction device preferably having a roughened surface at their distal end so that they can be gripped and oriented reliably by the end effector.

10. The liquid-jet scalpel of claim 1, wherein the nozzle and the suction device are arranged coaxially with respect to each other and are designed in a rotational symmetrical manner so that the joint only has one degree of freedom.
